(19) United States
(12) Patent Application Publication
(10) Pub. No.: US 2025/0019657 A1
(43) Pub. Date: Jan. 16, 2025

(54) Generation of Adipocytes from Bovine Embryonic Stem Cell Lines

(71) Applicant: Wisconsin Alumni Research Foundation, Madison, WI (US)

(72) Inventors: Igor I. Slukvin, Verona, WI (US); Nick Strelchenko, DeForest, WI (US)

(21) Appl. No.: 18/770,187

(22) Filed: Jul. 11, 2024

Related U.S. Application Data
(60) Provisional application No. 63/513,412, filed on Jul. 13, 2023.

(51) Int. Cl. C12N 5/077

(52) U.S. Cl. CPC C12N 5/0653; C12N 2501/16; C12N 2501/235; C12N 2501/415; C12N 2501/602; C12N 2501/603; C12N 2501/727; C12N 2501/734; C12N 2506/02; C12N 2533/50; C12N 2533/54

(57) ABSTRACT

Methods for generating feeder-free and serum-free bovine embryonic stem cells (bESCs) and meat components, such as adipocytes, from bESCs are provided along with the related compositions.

### CROSS-REFERENCE TO RELATED APPLICATION

This application claims priority to U.S. Provisional Application No. 63/513,412, filed Jul. 13, 2023, which is incorporated by reference in its entirety.

FIELD OF THE DISCLOSURE

The present disclosure generally relates to methods, cells, and compositions for preparing bovine embryonic stem cells (bESCs) and adipocytes from bESCs. In particular, provided herein are methods for generating feeder-free and serum-free bovine embryonic stem cells (bESCs) and adipocytes from bESCs along with the related compositions.

### BACKGROUND OF THE DISCLOSURE

By virtue of their capacity for pluripotent differentiation and indefinite self-renewal, pluripotent stem cells (PSCs) including bovine embryonic stem cells (bESCs) represent an ideal starting material for a wide range of cellular production processes, including cellular agriculture to generate meat products in vitro without harming or killing live animals while simultaneously reducing land resources for farming. However, advancing stem cell technologies for cellular agriculture requires development of protocols for culture and differentiation of PSCs from domesticated animals in chemically-defined conditions without any animal serum, xenogeneic feeders, or other components.

Meat is composed of skeletal muscle cells (˜90%), adipose and connective tissues (˜10%), and blood (˜0.3%) (Ben-Arye et al., Tissue Engineering for Clean Meat Production. Frontiers in Sustainable Food Systems. 2019, 3). Theoretically, these major components of meat could each be generated directly from bESCs. However, reliable sources of bESCs and methodological approaches to leverage such bESCs for meat component manufacturing (e.g., adipocyte, fibroblast, and skeletal muscle cell production) are required to reach commercially viable levels of production.

### BRIEF SUMMARY OF THE DISCLOSURE

The present disclosure describes methods, cells, and compositions for preparing bovine embryonic stem cells (bESCs) and adipocytes from bESCs.

In one aspect, the present disclosure provides a method of culturing bovine embryonic stem cells (bESCs), the method includes the steps of a) culturing bESCs in a serum-free culture medium comprising an effective amount of a γ-secretase inhibitor on a matrix-coated culture vessel in feeder-free conditions; and b) obtaining an expanded population of bESCs expressing Oct4, Sox2, and alkaline phosphatase.

In one embodiment of the first aspect, the γ-secretase inhibitor comprises (2S)-N-[(3,5-Difluorophenyl)acetyl]-L-alanyl-2-phenyl]glycine 1,1-dimethylethyl ester (DAPT).

In one embodiment of the first aspect, the matrix comprises vitronectin or collagen.

In a second aspect, the present disclosure provides a method of producing adipocytes from bovine embryonic stem cells (bESCs), the method includes the steps of (a) culturing bESCs in a serum-free culture medium on a matrix-coated cell culture vessel in feeder-free conditions to obtain bESCs expressing Oct4, Sox2, and alkaline phosphatase, wherein the cell culture medium comprises an effective amount of γ-secretase inhibitor; and (b) dissociating the bESCs and culturing the dissociated cells on a matrix-coated cell culture vessel under serum-free medium to obtain adipocytes.

In one embodiment of the second aspect, the γ-secretase inhibitor comprises DAPT.

In one embodiment of the second aspect, the serum-free culture medium in step (a) is mTeSR medium.

In one embodiment of the second aspect, the matrix comprises vitronectin or collagen.

In one embodiment of the second aspect, the dissociated cells are cultured in serum-free medium for 3-5 passages.

In one embodiment of the second aspect, the serum-free medium in step (b) is Gibco StemPro™ MSC SFM.

In one embodiment of the second aspect, the adipocytes exhibit adipocyte morphology and accumulate lipids.

In one embodiment of the second aspect, the adipocytes express adiponectin and leptin.

In a third aspect, the present disclosure provides a method of producing adipocytes from bovine embryonic stem cells (bESCs), the method includes the steps of (a) culturing blastocysts on mouse embryonic fibroblasts (MEFs) in a serum-free culture medium comprising an effective amount of one or more inhibitors of the WNT pathway to obtain bESCs expressing Oct4, Sox2, and alkaline phosphatase; (b) culturing the bESCs from step (a) on a matrix-coated cell culture vessel in feeder-free conditions to form bESC spheres; and (c) dissociating cells from the bESC spheres and culturing the dissociated cells on a matrix-coated cell culture vessel under serum-free medium to obtain adipocytes.

In one embodiment of the third aspect, the blastocysts are cultured on MEFs for 12-14 days.

In one embodiment of the third aspect, the one or more inhibitors of the WNT pathway comprises IWR-1, CCT251545, and/or iCRT3.

In one embodiment of the third aspect, the culture medium further comprises an Lck/Src inhibitor, Activin A, and LIF. In one embodiment, the Lck/Src inhibitor comprises WH-4-023.

In one embodiment of the third aspect, the matrix comprises vitronectin or collagen.

In one embodiment of the third aspect, the bESCs in step (b) are cultured for about 5 days.

In one embodiment of the third aspect, the dissociated cells are cultured in serum-free medium for 3-5 passages.

In one embodiment of the third aspect, the serum-free medium in step (c) is Gibco StemPro™ MSC SFM.

In one embodiment of the third aspect, the adipocytes exhibit adipocyte morphology and accumulate lipids.

In one embodiment of the third aspect, the adipocytes express adiponectin and leptin.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1 shows derivation of bESCs and their maintenance according to Methods 1-3. Images demonstrate sequential steps in the derivation of bESCs and growing them according to Methods 1, 2, and 3 on plates precoated with vitronectin, as indicated.

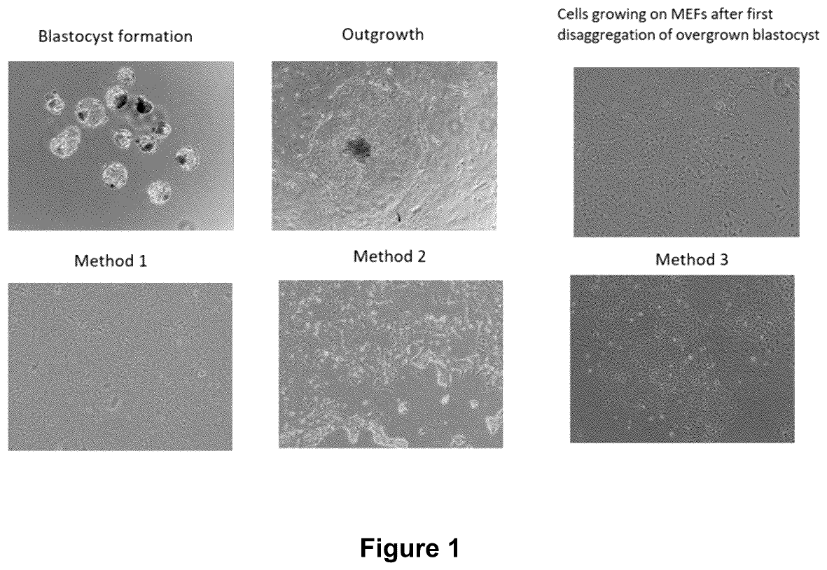

FIG. 2 shows bESC growth on different matrices. bESCs were generated using Method 1 and cultured in medium composed mTeSR™Plus, 4 mM Glutamax, 1/100 dilution of Chemically Defined Lipid Concentrate (CDLC; ThermoFisher #11905031), and 2.5 μM β-catenin inhibitor IWR-1 on cell culture plate precoated with Matrigel®, Matrigel®/gelatin, Collagen or gelatin as indicated. Matrigel® and/or gelatin do not support the growth of bESCs. Collagen supports bESC cultures. However, bESC attachment to collagen is weak.

FIG. 3 shows the growth of bESCs in mTeSR™Plus medium with DAPT and IWR-1 combinations at different concentrations.

FIG. 4 shows the expression of Sox2, Oct4, and alkaline phosphatase in bESCs derived and growing according Method 1.

FIG. 5 shows the expression of Sox2, Oct4, and alkaline phosphatase in bESCs according to Method 2.

FIG. 6 shows the expression of Sox2, Oct4, and alkaline phosphatase in bESCs growing according to Method 3.

FIG. 7 shows the formation of floating spheroid colonies in overgrown bESC cultures from Method 1 and Method 2.

FIG. 8 illustrates steps for generation of adipocytes from bESCs obtained according to Method 1 and Method 2.

FIG. 9 shows adipogenic differentiation of bESCs in different media.

FIG. 10 demonstrates the expression of Leptin and Adiponectin (ADIPOQ) expression in bESC-derived adipocytes.

FIG. 11 demonstrates the generation of adipocytes from bESCs maintained according to Method 3.

### DETAILED DESCRIPTION

It is to be understood that the particular aspects of the specification described herein are not limited to the specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements, or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indictates otherwise.

As utilized in accordance with the present disclosure, unless otherwise indicated, all technical and scientific terms shall be understood to have the same meaning as commonly understood by one of ordinary skill in the art.

In some embodiments, numerical percentages, ranges, or amounts of substances disclosed herein can vary in amount by ±1, 3, 5, 10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the instant invention.

### Overview

Although numerous studies described generation of meat components from stem cells, they typically use muscle stem cells from animals, known as satellite stem cells (Post, M. J. Cultured meat from stem cells: challenges and prospects. Meat Sci 92, 297-301 (2012); Stanton et al., Prospects for the Use of Induced Pluripotent Stem Cells in Animal Conservation and Environmental Protection. Stem Cells Transl Med 8, 7-13 (2019)). However, satellite stem cells have limited expansion and differentiation potentials which requires constant supply of these cells from animals in order to manufacture meat and use different cell sources for production of other cellular components of meat. In addition, many described protocols rely on animal serum for satellite stem cell expansion and differentiation. Only one study describes generation of skeletal muscles from porcine pluripotent stem cells (PSCs) (Genovese et al., Enhanced Development of Skeletal Myotubes from Porcine Induced Pluripotent Stem Cells. Sci Rep. 2017; 7:41833). However, that described protocol uses cells genetically modified with lentivirus and still requires fetal bovine serum for proliferation and, therefore, cannot be considered as entirely animal free. Hence, so far, production of meat components from bovine PSCs has not been described.

The present disclosure describes the generation of bovine embryonic stem cells (bESCs), the method of expanding bESC populations in defined serum-free and feeder-free conditions, and the method of differentiating bESCs into major meat components, including adipocytes (fat-producing cells).

The embodiments of the present disclosure were envisioned at least in part based on the discoveries discussed below. Methods for generation of bESCs, their expansion in feeder- and serum-free conditions, and methods for generation of adipocytes from bESCs are described.

Without intending to limit the disclosure, several embodiments of the disclosure are described below for purpose of illustration.

The term “feeder-free” describes a cell culture condition in which a layer of feeder cells is omitted during the culturing of a given cell population.

The term “expansion” describes culturing cells to promote growth without differentiation.

The term “coating” refers to a surface treatment to a cell culture vessel that promotes an intended cell culture outcome, such as expansion and/or or differentiation of the cultured cells. For example, cells cultured on a treated surface are in contact with and growing on top on a cell culture vessel surface that is coated with one or more materials that promote attachment of cells to the surface. The coating materials can include glycoproteins such as vitronectin, or other extracellular proteins such as collagen, gelatin, or a combination thereof such as Matrigel®. In another example, the cell culture vessel can be coated with a layer of cells (feeder cells).

In one aspect, the present disclosure provides a method of culturing bovine embryonic stem cells (bESCs), the method includes the steps of a) culturing bESCs in a serum-free culture medium comprising an effective amount of a γ-secretase inhibitor on a matrix-coated culture vessel in feeder-free conditions; and b) obtaining an expanded population of bESCs expressing Oct4, Sox2, and alkaline phosphatase.

In one embodiment of the first aspect, the γ-secretase inhibitor comprises (2S)-N-[(3,5-Difluorophenyl)acetyl]-L-alanyl-2-phenyl]glycine 1,1-dimethylethyl ester (DAPT). Other examples of γ-secretase inhibitors that can be used herein include, for example, L-658,458 (transition state mimic), IL-X (cbz-IL-CHO), WPE III-31-C, Compound E, Sulfonamide, JLK6, Sulindac sulfide, Indomethacin, Estrogen, and RO4929097, as described in Zhavoronkov et al., (J Mol Med (2012) 90:1361-1389) as well as avagacestat, BMS 299897, DBZ, LY 450139, MRK 560, and PF 3084014 hydrobromide, which are available from Tocris Bioscience (e.g., see www.tocris.com/pharmacology/gamma-secretase/inhibitors).

In one embodiment of the first aspect, the matrix comprises vitronectin or collagen.

In a second aspect, the present disclosure provides a method of producing adipocytes from bovine embryonic stem cells (bESCs), the method includes the steps of (a) culturing bESCs in a serum-free culture medium on a matrix-coated cell culture vessel in feeder-free conditions to obtain bESCs expressing Oct4, Sox2, and alkaline phosphatase, wherein the cell culture medium comprises an effective amount of γ-secretase inhibitor; and (b) dissociating the bESCs and culturing the dissociated cells on a matrix-coated cell culture vessel under serum-free medium to obtain adipocytes.

In one embodiment of the second aspect, the γ-secretase inhibitor comprises DAPT.

In one embodiment of the second aspect, the serum-free culture medium in step (a) is mTeSR medium.

In one embodiment of the second aspect, the matrix comprises vitronectin or collagen.

In one embodiment of the second aspect, the dissociated cells are cultured in serum-free medium for 3-5 passages.

In one embodiment of the second aspect, the serum-free medium in step (b) is Gibco StemProT™ MSC SFM.

In one embodiment of the second aspect, the adipocytes exhibit adipocyte morphology and accumulate lipids.

In one embodiment of the second aspect, the adipocytes express adiponectin and leptin.

In a third aspect, the present disclosure provides a method of producing adipocytes from bovine embryonic stem cells (bESCs), the method includes the steps of (a) culturing blastocysts on mouse embryonic fibroblasts (MEFs) in a serum-free culture medium comprising an effective amount of one or more inhibitors of the WNT pathway to obtain bESCs expressing Oct4, Sox2, and alkaline phosphatase; (b) culturing the bESCs from step (a) on a matrix-coated cell culture vessel in feeder-free conditions to form bESC spheres; and (c) dissociating cells from the bESC spheres and culturing the dissociated cells on a matrix-coated cell culture vessel under serum-free medium to obtain adipocytes.

In one embodiment of the third aspect, the blastocysts are cultured on MEFs for 12-14 days.

In one embodiment of the third aspect, the one or more inhibitors of the WNT pathway comprises IWR-1, CCT251545, and/or iCRT3.

In one embodiment of the third aspect, the culture medium further comprises an Lck/Src inhibitor, Activin A, and LIF. In one embodiment, the Lck/Src inhibitor comprises WH-4-023.

In one embodiment of the third aspect, the matrix comprises vitronectin or collagen.

In one embodiment of the third aspect, the bESCs in step (b) are cultured for about 5 days.

In one embodiment of the third aspect, the dissociated cells are cultured in serum-free medium for 3-5 passages.

In one embodiment of the third aspect, the serum-free medium in step (c) is Gibco StemPro™ MSC SFM.

In one embodiment of the third aspect, the adipocytes exhibit adipocyte morphology and accumulate lipids.

In one embodiment of the third aspect, the adipocytes express adiponectin and leptin.

In some embodiments, the present disclosure provides an expanded population of bESCs cultured in a serum-free culture medium comprising an effective amount of a γ-secretase inhibitor. The bESCs are cultured on a matrix-coated culture vessel in feeder-free conditions. The bESCs express Oct4, Sox2, and alkaline phosphatase.

In some embodiments, bESC populations obtained according to the methods disclosed herein can express Oct4, Sox2, and varied levels of alkaline phosphatase. For example, alkaline phosphatase can be highly expressed, weakly expressed, or partially expressed in bESC cells.

In some embodiments, the present disclosure provides a population of adipocytes cultured from bESCs in a serum-free culture medium on a matrix-coated cell culture vessel in feeder-free conditions. The adipocytes exhibit adipocyte morphology and accumulate lipids. In some embodiments, the adipocytes express adiponectin and leptin.

In some embodiments, the present disclosure is directed to a meat component cultured in vitro from bESCs. The meat component comprises an adipocyte that accumulates lipids and expresses adiponectin and/or leptin.

In some embodiments, the present disclosure is directed to a meat component cultured in entirely animal free conditions from bESCs. The meat component comprises an adipocyte that accumulates lipids and expresses adiponectin and/or leptin.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Generation of Bovine ESCs (bESCs) From Blastocysts and Expansion of bESCs on Feeder-Free Conditions

Several protocols have been described for differentiation of bovine pluripotent stem cells (PSCs). Originally, serum-containing media conditions and culture on murine embryonic fibroblasts (MEFs) feeder cells were used in these protocols (Saito et al., Bovine embryonic stem cell-like cell lines cultured over several passages. Rouxs Arch Dev Biol. 1992, 201(3):134-141; Stice et al., Pluripotent bovine embryonic cell lines direct embryonic development following nuclear transfer. Biol Reprod. 1996, 54(1):100-110; and Forsberg et al., Production of cloned cattle from in vitro systems. Biol Reprod. 2002, 67(1):327-333). Recent protocols for bESC derivation use MEFs and serum-free mTeSR™Plus medium supplemented with 20 ng/ml human FGF2 and 2.5 μM IWR1 (Bogliotti et al., Efficient derivation of stable primed pluripotent embryonic stem cells from bovine blastocysts. Proc Natl Acad Sci U S A. 2018, 115(9):2090-2095; and Soto et al., Simplification of culture conditions and feeder-free expansion of bovine embryonic stem cells. Sci Rep. 2021, 11(1):11045), or mTeSR™ supplemented with 1 μM CHIR99021, 0.3 μM WH-4-023, 5 μM XAV939 or 5 μM IWR-1, 50 μg/mL Vitamin C, 10 ng/mL LIF, and 20.0 ng/ml Activin A (Zhao et al., Establishment of bovine expanded potential stem cells. Proc Natl Acad Sci U S A. 2021, 118(15)). In their studies, Soto et al. revealed that bESCs obtained on MEFs can be expanded in feeder-free conditions using plates precoated with vitronectin and N2B27 medium consisting of a mixture of 1:1 DMEM/F12 medium and Neurobasal medium, 0.5% v/v N-2 Supplement, 1% v/v B-27 Supplement, 2 mM MEM Non-Essential Amino Acid Solution, 1% v/v GlutaMAX Supplement (35050-061, Gibco), 0.1 mM 2-mercaptoethanol, 100 U/mL Penicillin and 100 μg/mL Streptomycin (15140-122, Gibco).

The experiments described below employed several Methods with modified coating and medium conditions to achieve populations of bESCs that retain their cellular identity after differentiation from precursors, and maximize their expansion.

I. Derivation of bESCs From Blastocysts
a) Culture of Blastocysts on Murine Embryonic Fibroblasts (MEF) Generated Stable bESC Cell Lines

bESCs were generated from IVM/IVF bovine embryo derived blastocysts on 6th to 7th days post-fertilization. Briefly, bovine embryos were obtained from an outside source and cultured in TeSR Plus medium supplemented with 15% fetal bovine serum from 24 to 72 hours to get an expanded blastocyst stage. After that zona pellucida was removed by treating embryos with pronase. Embryos with stripped zona pellucida were plated on irradiated MEFs (obtained from WICell) in a 4-well IVF plate and cultured according to Method 1 or Method 2. Following expansion of inner cell mass after culture for 2-3 weeks on MEFs, cells were disaggregated by TrypLE, and bESC were generated from individual blastocysts by two Methods (Method 1 or Method 2) to maximize efficiency.

To determine if feeder-free conditions were suitable for maximizing generation of bESCs from inner cell mass, individual blastocysts were placed into culture plates according to Method 1 or Method 2 with coating conditions modified as shown in Table 1. Coating conditions using MEF feeder cells or vitronectin (feeder-free) were tested. In the first set, 11 bovine embryos were used to establish 4 bESC lines, 3 of which were differentiated and tossed after passage 5-7. Only one cell line remained undifferentiated and maintained expression of bESC pluripotency factors Oct4 and Sox2. Using this approach, culture of disaggregated inner cell mass on MEFs appears to be required, as no bESC line was generated following culture on extracellular matrix vitronectin. However, bESCs were generated when inner cell mass cells formed colonies on MEFs (P0) and subsequently (P1 or P2) transferred and cultured on vitronectin in bESC media.

TABLE 1
Generation of bESC lines using different methods.

| | Number of | | Number of bESC |
|---|---|---|---|
| Coating | Embryos | Method | lines generated |
| MEF | 11 | 1 | 1 |
| vitronectin | 12 | 1 | 0 |
| vitronectin + Cover | 10 | 1 | 0 |
| Glass on the top | | | |
| MEF | 14 | 2 | 2 |
| MEF | 27 | 2 | 12 |

In Method 1, individual blastocysts were seeded on MEFs or vitronectin and cultured in bESC medium 1 described in Table 2. Following blastocyst attachment and outgrowth formation, blastocysts cultured for 12 to 14 days were treated with TrypLE (Gibco, Cat #12563-029) and marked as Passage 0 (P0). The timing for TrypLE treatment appears to be important and cannot exceed more than 3 min at +37° C., after that TrypLE was aspirated and replaced with cold DMEM medium. Cell disaggregation into small clumps was performed using a dissection microscope with a glass pipette and syringe with a 25G needle. Following disaggregation, cells were cultured on MEFs in medium 1 for 1 or 2 passages. Subsequently cells were transferred into plates in feeder-free conditions, coated with different matrices and cultured in bESC Medium 1. bESC were passaged with TrypLE when cultured on MEFs and PBS-EDTA when cultured in feeder-free conditions with cell culture vessels coated with vitronectin, collagen, or a combination collagen and fibronectin.

TABLE 2
bESC culture medium 1.

| | | |
|---|---|---|
| Composition | Volume | Final Concentration |

| mTeSR1 Plus | 50 | mL | | |
|---|---|---|---|---|
| 200 mM GlutaMAX | 1 | mL | 4 | mM |
| CDLC | 0.5 | mL | 1:100 | |
| 2.5 mM IWR-1* | 50 | μL | 2.5 | μM |

CDLC—Chemically Defined Lipid Concentrate (Thermofisher Cat#11905031).
*Preparation stock of IWR-1 (Sigma; Cat#I0161) were obtained by dissolving 4.2 mg IWR-1 in 4.1 mL of DMSO to get a 2.5 mM or 1000X stock solution.

In Method 2, individual IVM/IVF derived blastocysts were placed on MEFs in bESC medium 2 described in Table 3. Following blastocyst attachment and outgrowth formation, blastocysts cultured for 12 to 14 days were treated with TrypLE (Gibco, Cat #12563-029) and marked as Passage 0 (P0). The timing for TrypLE treatment appears to be important and cannot exceed more than 3 min at +37° C., after that TrypLE was aspirated and replaced with cold DMEM medium. Cell disaggregation into small clumps was performed under a dissection microscope with glass pipette and syringe with a 25G needle. Following disaggregation, cells were cultured on MEFs in medium 2 for 1 or 2 passages. Subsequently cells were seeded in plates in feeder-free conditions, coated with different matrices and cultured in bESC medium 2. bESC were passaged with PBS-EDTA.

TABLE 3
bESC culture medium 2.

| | | |
|---|---|---|
| Composition | Volume | Final Concentration |

| mTeSR Plus | 50 | mL | | |
|---|---|---|---|---|
| 3 mM WH-4-023* | 5 | μL | 0.3 | μL |
| CDLC | 0.5 | mL | 1:100 | |
| 2.5 mM IWR-1 | 50 | μL | 2.5 | μM |
| 20 μg/μL Activin A** | 50 | μL | 20 | ng/mL |
| LIF 25 μg | 25 | μL | 10 | ng/mL |

*Preparation stock of WH-4-023 (MedChemExpress CCT251545; Cat# HY-12299; Lot#27962) 5 mg was dissolved in 2.93 mL DMSO to get a 10,000x or 3 mM solution, which was aliquoted in 50 μL amounts per vial and stored at −80° C.
**Preparation stock of Activin A (Peprotech Activin A; Cat# GMP120-14E; Lot#27962): 10 μg of Activin A was dissolved in 1 mL DPBS and aliquoted in 50 μL amounts per PCR tube; 1 PCR tube was sufficient for 50 mL mTeSR.

II. Expansion of bESCs in Feeder-Free and Serum-Free Conditions
a) Determining Cellular Matrix Coating Supporting Expansion of bESCs in Feeder-Free Condition

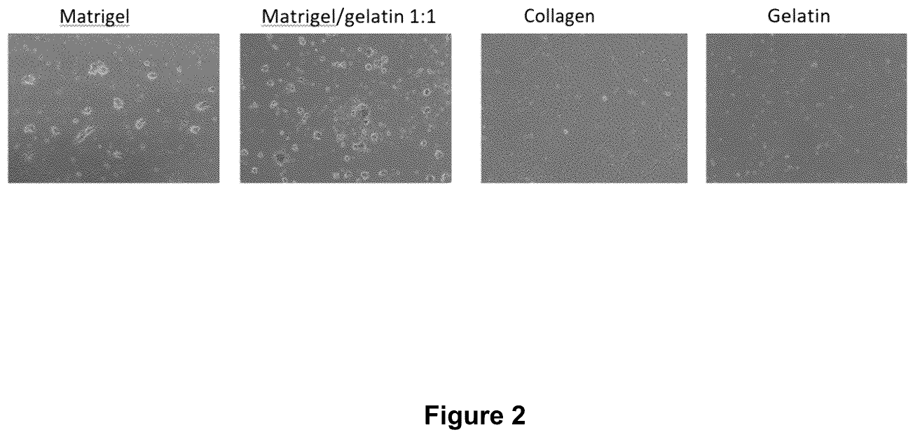

After co-culture with MEFs, bESCs were collected using TrypLE treatment and cultured in 6-well plastic plates in feeder-free conditions for expansion. Different matrix proteins were used to pre-coat the plastic plates, including Vitronectin XF, Collagen, Matrigel®, gelatin, myMATRIX IPSC (denovoMATRIX), and a combination of Matrigel® to gelatin in a 1:1 ratio, or on naked plastic and cultured in bESC medium 1 or 2. bESCs did not attach to naked plastic. Although bESCs attached to plates pre-coated with gelatin, Matrigel®, gelatin/Matrigel® or myMATRIX, they did not grow. bESCs growing on collagen and vitronectin maintained a typical bESC morphology. However, we noted that collagen did not work well with all media. bESCs adhered to collagen very lightly when growing in bESC medium 1 (FIG. 2). When bESC medium 2 was used, cells were able to form colonies and grow on collagen. Thus, we concluded that Vitronectin XF is the best matrix to support growth of bESCs in feeder-free conditions. However, collagen pre-coated plates can be used to support bESC growth in bESC medium 2. FIG. 2 demonstrates the growth of bESCs on different matrices in bESC medium 1.

b) Other Modifications in Medium Conditions Supporting Expansion of bESCs in Feeder-Free Condition

1) Wnt-inhibitors (Medium la and Medium 1b): In prior studies, mTeSR Plus medium supplemented with 20 ng/ml human FGF2 and 2.5 μM IWR1 was used for bESC cultures (Saito et al. 1992; Stice et al. 1996). To determine whether IWR1 can be substituted with other Wnt/β-catenin signaling inhibitors, bESC growth in Method 1 was tested in TeSR medium with other Wnt inhibitors, including: 1) iCRT-3, an oxazole compound that binds to the Arg469 residue on β-catenin and inhibits the interaction of β-catenin-TCF4 complex 10; and 2) orally bioavailable CCT251545, which inhibits Wnt, GSK3α, and human mediator complex-associated protein kinases CDK8 and CDK19 including expression of genes regulated by STAT1 and weakly inhibits of tankyrase enzymes (Dale et al., A selective chemical probe for exploring the role of CDK8 and CDK19 in human disease. Nat Chem Biol 11, 973-980 (2015); and Mallinger et al., Discovery of potent, orally bioavailable, small-molecule inhibitors of WNT signaling from a cell-based pathway screen. J Med Chem 58, 1717-1735 (2015)). bESC media compositions are shown in Table 4 and Table 5.

bESC growth in Medium 1 was tested with concentrations of 1 nM, 5 nM, or 10 nM iCRT3 (Medium 1a). Concentrations 1 nM and 5 nM did not support bESC growth on Vitronectin and cells died after 1-2 passages (1 nM) or 2-4 passages (5 nM). iCRT3 at a concentration 10 nM supported bESC growth with typical morphology and expression of Sox2 and Oct4 for more than 10 passages.

In another set of experiments, bESC growth in Medium 1 was tested with concentrations of 1 nM, 5 nM, 75 nM, or 150 nM CCT251545 (Medium 1b). Concentrations of 1 nM, 5 nM, and 75 nM did not support bESC growth on vitronectin and cells died after 2-3 passages (1 nM). CCT251545 at a concentration 150 nM supported bESC growth with typical morphology and expression of Sox2 and Oct4 for more than 7 passages.

TABLE 4
bESC culture medium 1a.

| | | |
|---|---|---|
| Composition | Volume | Final Concentration |

| mTeSR Plus | 50 | mL | | |
|---|---|---|---|---|
| 200 mM GlutaMAX | 1 | mL | 4 | mM |
| CDLC | 0.5 | mL | 1:100 | |
| 0.1 mM iCRT3* | 5 | μL | 10 | nM |

*Preparation stock of iCRT3 (MedChemExpress 5 mg vial; Cat# HY-103705, Lot#27962; MW = 394.5 g/mol): 5 mg iCRT3 was dissolved in a total volume of exactly 1.27 mL DMSO in order to get the concentration of 10 mM. To get a working stock, 10 μL of 10 mM solution was diluted in 1 mL DMSO.

TABLE 5
bESC culture medium 1b.

| | | |
|---|---|---|
| Composition | Volume | Final Concentration |

| mTeSR Plus | 50 | mL | | |
|---|---|---|---|---|
| 200 mM GlutaMAX | 1 | mL | 4 | mM |
| CDLC | 0.5 | mL | 1:100 | |
| 150 μM CCT251545* | 5 | μL | 150 | nM |

*Preparation stock of CCT251545 (MedChemExpress CCT251545; Cat# HY-12681, Lot#20068). Dissolve vial containing 5 mg of solute (MW = 422 g/mol) in a total solution volume of exactly 1.58 mL DMSO. Take 20 μL of this solution and add to 1 mL of DMSO to make 1000X solution with concentration of 150 μM.

2) DAPT in mTeSR medium (Medium 3): DAPT: (2S)-N-[(3,5-Difluorophenyl)acetyl]-L-alanyl-2-phenyl]glycine 1,1-dimethylethyl ester is a γ-secretase inhibitor which blocks NOTCH signaling. γ-secretase inhibition also affects the cleavage of amyloid precursor proteins, E-cadherin, ErbB4, CD44, tyrosinase, TREM2, and alcadein (Zhang et al., The g-secretase complex: from structure to function. Front. Cell. Neurosci. 8:427 (2014). DAPT has been used to induce neuronal differentiation of human ESCs (Crawford et al., The notch response inhibitor DAPT enhances neuronal differentiation in embryonic stem cell-derived embryoid bodies independently of sonic hedgehog signaling. Dev Dyn 236, 886-892 (2007)) and long-term culture of hepatocytes (Xiang et al., Long-term functional maintenance of primary human hepatocytes in vitro. Science 364, 399-402 (2019)). It has been also reported that inhibition of NOTCH signaling improves formation of human ESC colonies (Yu, X., Zou, J., Ye, Z., Hammond, H., Chen, G., Tokunaga, A., Mali, P., Li, Y. M., Civin, C., Gaiano, N., and Cheng, L. (2008). Notch signaling activation in human embryonic stem cells is required for embryonic, but not trophoblastic, lineage commitment. Cell Stem Cell 2, 461-471. 10.1016/j.stem.2008.03.001).

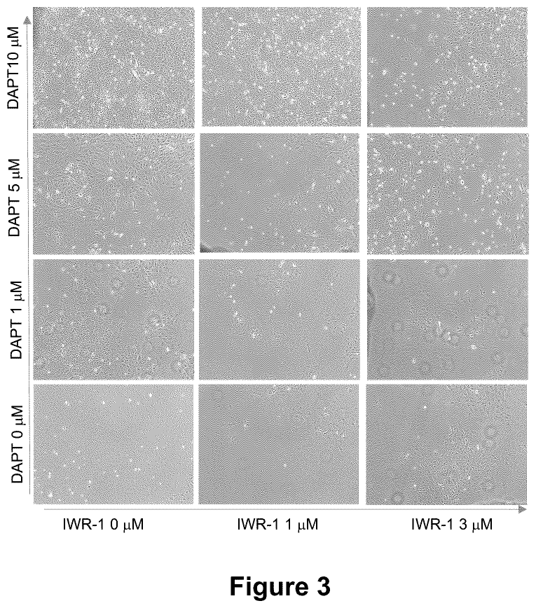

To test whether DAPT can be used for bESC cultures, the growth of bESC was tested in mTeSR medium with different concentrations of DAPT and IWR-1. Addition of DAPT alone at concentrations 5-10 μM to mTeSR medium was sufficient to support growth of bESCs. Addition of IWR-1 to DAPT had no effect on bESCs. (FIG. 3, Table 6; bESC Medium 3/Method 3). mTeSR with 10 μM DAPT alone supported bESC cultures for more than 7 passages.

TABLE 6
bESC culture medium 3.

| | | |
|---|---|---|
| Composition | Volume | Final Concentration |

| mTeSR Plus | 50 | mL | | |
|---|---|---|---|---|
| 200 mM GlutaMAX | 1 | mL | 4 | mM |
| CDLC | 0.5 | mL | 1:100 | |
| 10 mM DAPT* | 50 | μL | 10 | μM |

*Preparation stock of DAPT (Sigma DAPT; Cat# D5942). Dissolve 10 mg of DAPT in 2.31 mL DMSO to get 1,000X or 10 mM concentration. Aliquot 50 μL/tube and store at −80° C.

### Example 2: Evaluation of Pluripotency Markers in bESCs Cultured in Different Conditions

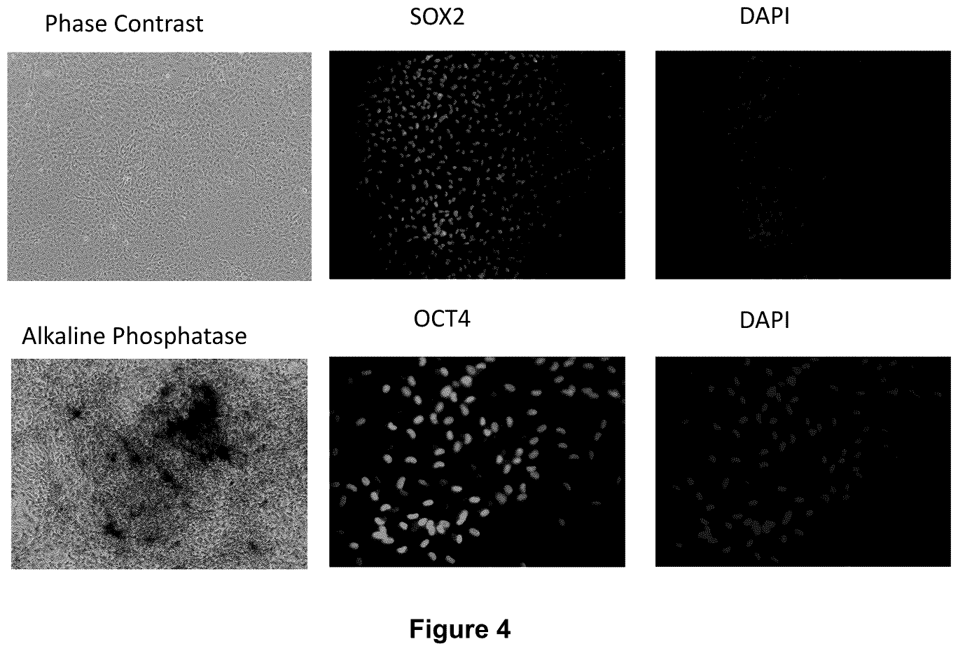

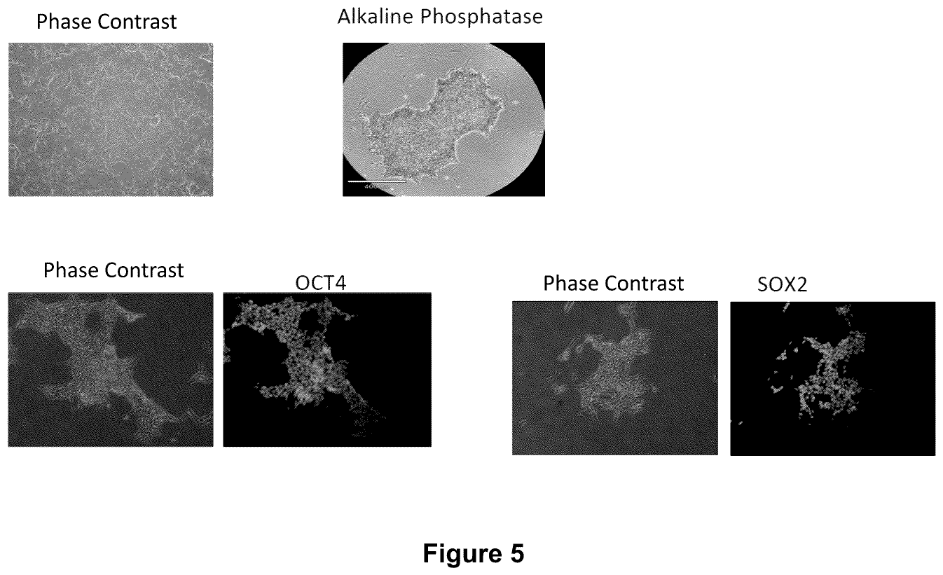

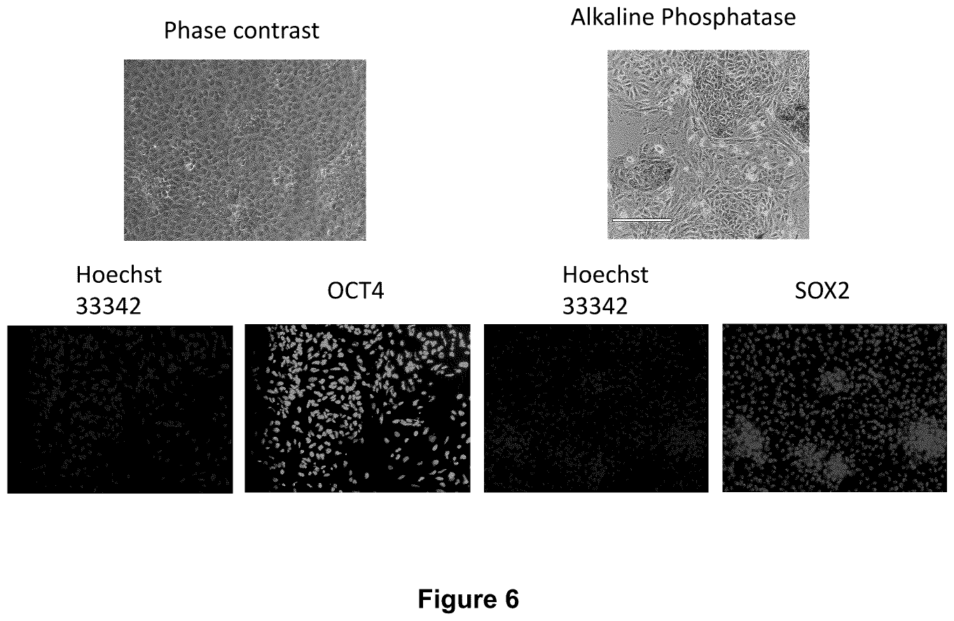

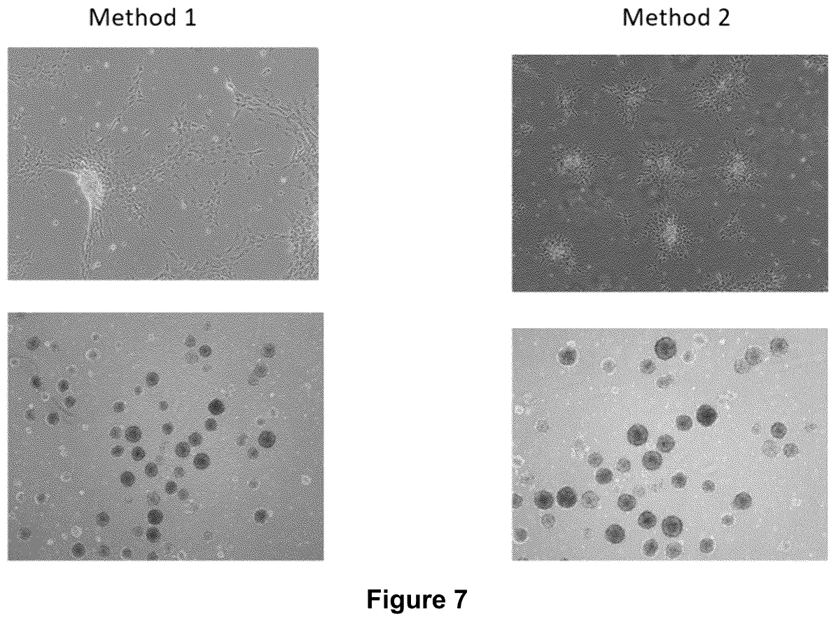

Typical morphology and expression of pluripotency markers Oct4, Sox2, and alkaline phosphatase in bESCs growing in different conditions are shown in FIGS. 4-6. bESCs generated and expanded using Methods 1-3 expressed typical ESC markers Oct4, Sox2, and alkaline phosphatase (FIGS. 4-6). However, some differences in alkaline phosphatase expression were observed between bESCs growing according different methods. While bESCs growing according to Method 2 demonstrated high levels of alkaline phosphatase expression (FIG. 5), bESCs growing according to Method 1 and Method 3 expressed alkaline phosphatase weakly and partially (FIGS. 4 and 6), respectively, suggesting that bESCs growing in these conditions acquire some features of primed epiblast stem cells. Noticeably, in cultures with high cell density under Methods 1 and 2 but not Method 3, bESCs formed spheres of approximately 150-250 μm in size. The spheres initially were lightly attached to cells clusters and eventually became detached and floated free in dishes (FIG. 7).

The differences in morphology, expression of pluripotency markers, and capacity of spontaneous spheroid formation by bESCs cultured according to different methods are summarized in Table 7.

TABLE 7
Comparison of different bESCs and their
derivatives for differentiation.

| | | | | | Spontaneous |
|---|---|---|---|---|---|
| | | | | | formation |
| Method | Morphology | Oct 3/4 | Sox2 | AlkPhos | of Spheres |
| Method 1 | Monolayer | ++ | ++ | + | + |
| Method 2 | In colonies | ++ | ++ | ++ | + |
| Method 3 | Monolayer | ++ | ++ | +/− | − |

### Example 3: Generation of Adipocytes From bESCs

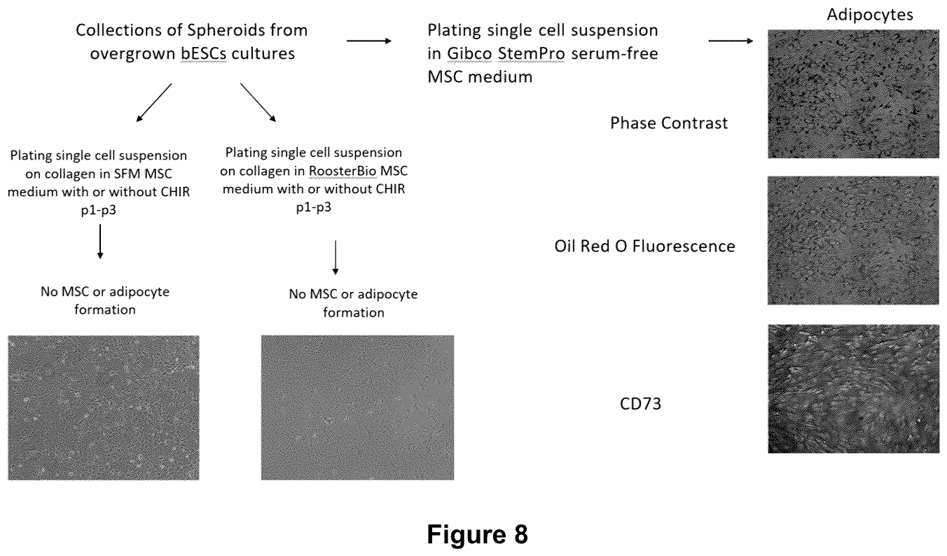

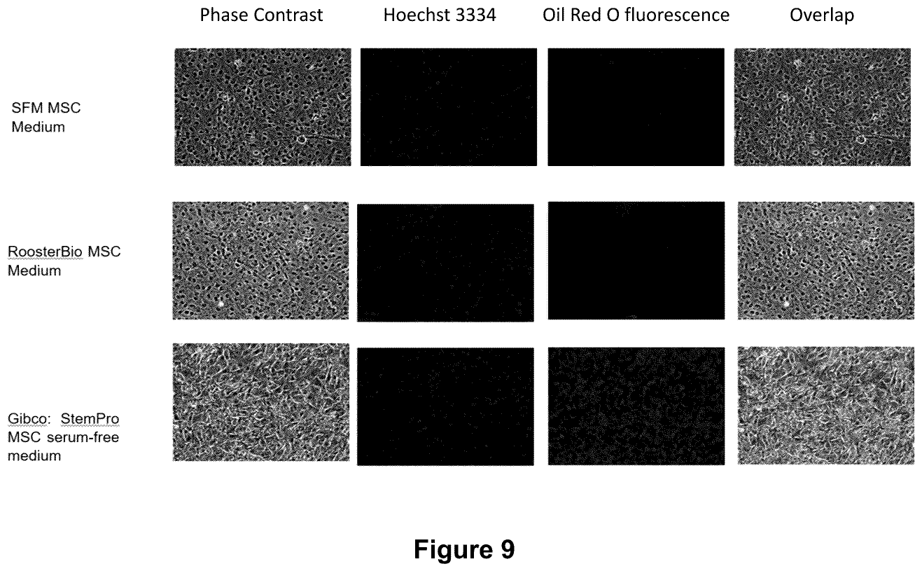

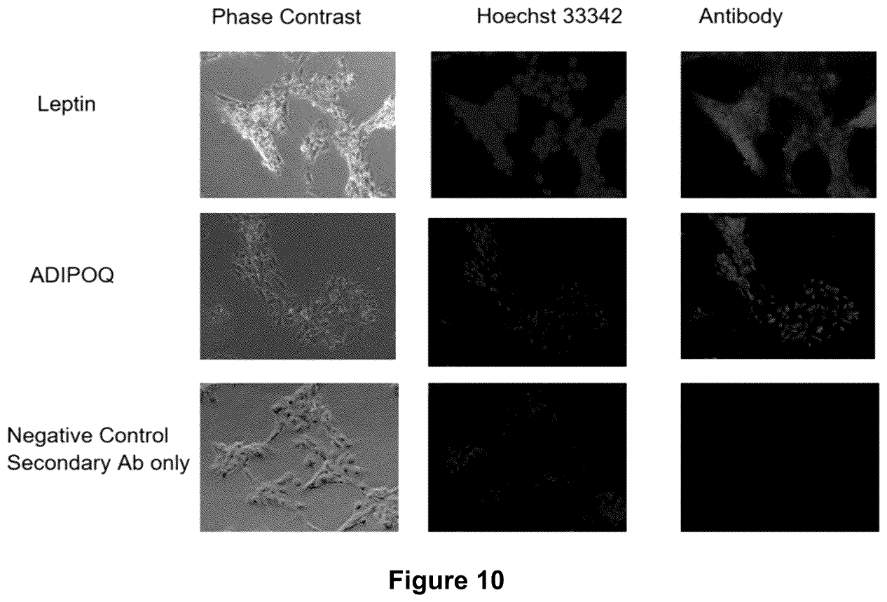

As noted above, in cultures at high density in Methods 1 and 2, bESCs formed spheres of approximately 150-250 μm in size. The spheres initially were lightly attached to cells clusters and eventually became detached and floated free in dishes (FIG. 7). For generation of adipocytes, spheres were collected from medium by soft centrifugation at 50 g for 3 min. After supernatant removal, sphere pellets were resuspended in enzyme free solution LeReSR (Stemcell, Cat #07174) at 37° C. for 3.5 min and diluted with cold mTeSR™Plus medium. 10⁵ dissociated cells were plated into each well of collagen-coated 6 well plates in Gibco StemPro™ MSC SFM (Cat #A1033201, and as described in Chase et al., (Stem Cells Transl Med. 2012 October; 1(10):750-8)) or serum-free MSFM medium developed in the our lab (Vodyanik, M. A., Yu, J., Zhang, X., Tian, S., Stewart, R., Thomson, J. A., and Slukvin, II (2010). A mesoderm-derived precursor for mesenchymal stem and endothelial cells. Cell Stem Cell 7, 718-729) with CHIR or without CHIR or RoosterBasal MSC CC MSC medium (RoosterBio). After 3-5 passages using TrypLE, cells growing in Gibco StemPro™ MSC SFM (Cat #A1033201) acquired adipocyte morphology, CD73 expression, and accumulated lipids (FIG. 8). Lipid accumulation within cells was confirmed by Oil Red O stain (FIG. 8). In contrast, cells growing in MSCF medium or RoosterBio medium failed to differentiate into MSCs with or without CHIR (FIGS. 8 and 9). In addition, adipocytes generated in Gibco StemPro™ MSC SFM expressed adipocyte markers ADIPOQ (adiponectin) and Leptin (FIG. 10).

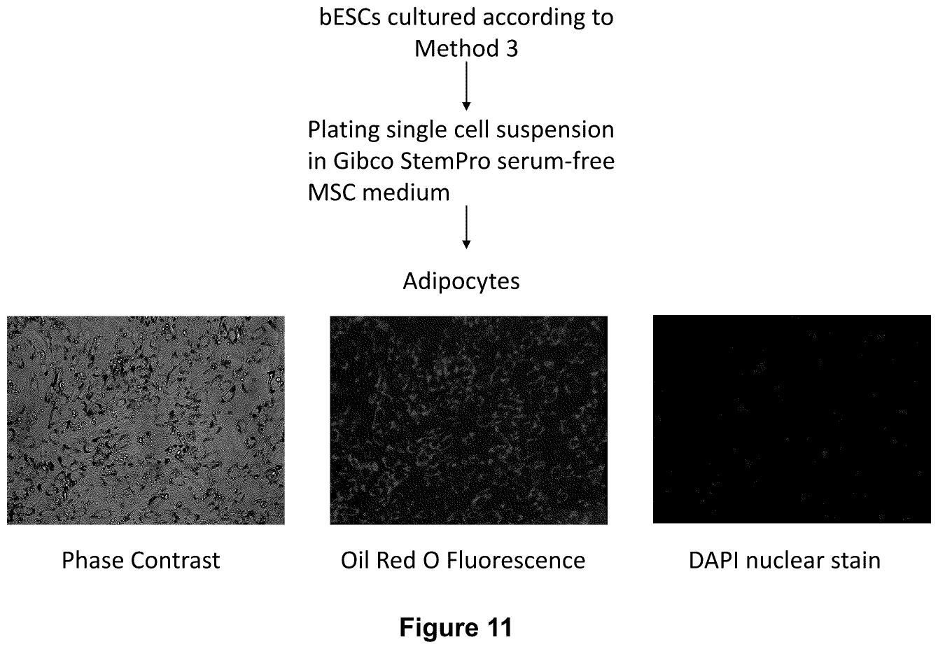

In another approach, we collected bESCs growing according to Method 3 and plated 10⁵ dissociated cells into collagen-coated 6 wells plate in Gibco StemPro™ MSC SFM (Cat #A1033201). After 1 passage using TrypLE, cells growing in Gibco StemPro™ MSC SFM acquired adipocyte morphology and accumulated lipids (FIG. 11).

### Materials and Methods

Derivation of bESCs From Blastocyst

IVM/IVF derived blastocysts on 6^{th} to 7^{th} days post-fertilization were obtained from Applied Reproductive Technology (www.appliedreproductivetechnology.com).

Obtained embryos cultured in TeSR Plus medium supplemented with 15% fetal bovine serum from 24 to 72 hours to get an expanded blastocyst stage. After that zona pellucida was removed by treating embryos with pronase.

To remove Zona pellucida, blastocysts were treated with preactivated (30 min at 37° C.) 3 mg/mL pronase solution (Sigma; Cat #P8811-100 mg) for 2-5 min. Dissolving of the zona pellucida was observed under microscope. Immediately after dissolution of the zona pellucida, embryos were washed with fresh PBS solution supplemented with 0.1% of bovine serum albumin, transferred into 4-wells plate seeded with MEFs and cultured in bESC Medium 1 composed of mTeSR™Plus, 4 mM Glutamax, 1/100 Chemically Defined Lipid Supplement (CDLC, Thermo Fisher #11905031) and 2.5 μM IWR-1. Alternatively, individual blastocysts after pronase treatment were cultured on MEFs in bESC Medium 2 composed of mTeSR™Plus (WiCell) supplemented with 2.5 μM IWR-1 (Sigma, Cat #I0161 25 mg) for inhibition of WNT pathway, 0.3 μM WH-4-023 as Lck/Src inhibitor (Sigma, Cat #SML1334), Activin A 20.0 ng/ml (R&D, Cat #338-AC-050) and 10 ng/ml of bovine (Kingfish Biotech, Cat #RP0997B-025) or human LIF (Millipore, Cat #LIF1010) was used. In another embodiment, bESC were cultured in bESC medium 3 composed of mTeSR™Plus (WiCell) supplemented with 10 mm of gamma γ-secretase inhibitor DAPT (2S)-N-[(3,5-Difluorophenyl)acetyl]-L-alanyl-2-phenyl]glycine 1,1-dimethylethyl ester.

Following blastocysts attachment and outgrowth formation (12 to 14 days of blastocyst culture on MEFs) dishes were treated with TrypLE (Gibco, Cat #12563-029) and marked as Passage 0. Timeframe for this treatment is critical and cannot be exceeded by more than 3.5 min at +37° C., after that TrypLE aspirated and replaced with cold DMEM medium. Cells disaggregation into small 50-100 μM clumps was performed under dissection microscope with glass pipette and syringe with 25 G needle. Following disaggregation cells were cultured on MEFs in Medium 1 or 2 for 1 or 2 passages.

### Determining Cellular Matrix Coating in Feeder-Free Condition

To establish feeder-free conditions for bESCs, bESCs were collected from coculture with MEFs using TrypLE treatment and transferred into 6-well plastic plates precoated Vitronectin XF, collagen, or Matrigel®.

Vitronectin Coating Preparation: A frozen vial of Vitronectin XF (2 mL) from Stem Cell Technology (Cat #07180) was thawed and mixed with 50 mL of DPBS and distributed into 6 of 6-well plates (˜1.4 mL/well) sealed with Parafilm and kept wrapped in foil at +4° C. for not more than 2 weeks. Before use, the plate was kept at +37° C. for at least 1 hr. Immediately after aspiration excess of liquid, bESCs in culture medium were added. Cells did not grow on dry or non-coated plastic surfaces.

Collagen Coating Preparation: Collagen (Sigma, Cat #5162-1G) was dissolved in distilled water and sterilized by filtration using 0.22 um filter. Collagen solution aliquoted into 70 μL PCR tubes and kept at −80° C. before use. For coating wells, the content of tube was transferred into 50 mL DPBS, carefully mixed, and distributed into 6 of 6 wells plate, approximately 1.3-1.4 mL/well. Plates sealed with Parafilm and kept at RT not more than 2 wecks.

Matrigel® Coating Preparation: Frozen stock (˜0.5 mL) of Matrigel® needs was thaw on ice and diluted in 50 mL of cold DPBS (+4° C.). Approximately 1.2-1.3 mL (9 μg/cm²) of diluted Matrigel® solution was added to each well. After 1 hour incubation at +37° C. excess Matrigel® was removed and wells washed with warm DPBS.

Maintenance of bESCs on Vitronectin

In Method 1, bESC colonies growing on Vitronectin were passaged by using TrypLe. Following medium aspiration 0.5-0.8 mL of TrypLE was added to each well of 6 well plate. Cells incubated for 3.5 min at 37.2° C. Then TrypLE has been carefully removed by aspiration and 1-1.2 mL of cold (+1-+4° C.) TeSRr medium was added, cells were suspended by pipette filtered via strainer (70 mkM), counted and an aliquot of 70,000 to 100,000 cell/well for 6 wells plate was taken and cells centrifuged at 100 g for 5 minutes. The cell pellet was resuspended in 9 mL growth medium and seeded on coated by vitronectin 6-well plate.

In Method 2, bESCs grew as loosely adherent colonies on collagen or Vitronectin-coated 6-well plate on and were passage using PBS-EDTA. Following aspiration of bESC growth media, 0.7-1.2 mL of PBS-EDTA solution was added to each well of 6 well plate. After incubation 5 to 6 min at +37° C., PBS-EDTA was carefully removed by aspiration and 1-1.2 mL of cold (+1-+4° C.) TeSR medium was added, cells were suspended by pipette, filtered via strainer (70 mkM), counted and an aliquot of 100,000 to 150,000 cell/well for 6 wells plate as taken and cells centrifuged at 100 g for 5 minutes. Cell pellets were resuspended in 9 mL growth medium and plated on by collagen or vitronectin coated 6-well plate. Cells grew for 5-7 days until they formed large colonies. PBS-EDTA solution was prepared by diluting 100 mL of concentrated PBS (Gibco Cat #70-011-069) with 900 mL deionized water and adding 1.5 mL of 0.5 M EDTA and sterilized by filtration.

In Method 3, bESC were cultured in bESC medium 3 composed of TeSR Plus (WiCell) supplemented with 10 mM of gamma γ-secretase inhibitor DAPT on collagen or a vitronectin-coated 6-well plate and were passage using PBS-EDTA. Following aspiration of bESC growth media, 0.7-1.2 mL of PBS-EDTA solution was added to each well of 6 well plate. After incubation 5 to 6 min at +37° C., PBS-EDTA was carefully removed by aspiration and 1-1.2 mL of cold (+1-+4° C.) TeSR medium was added, cells were suspended by pipette filtered via strainer (70 mkM), counted and an aliquot of 100,000 to 150,000 cell/well for 6 wells plate was taken and cells were centrifuged at 100 g for 5 minutes. Cell pellets were resuspended in 9 mL growth medium and plated on a collagen or vitronectin coated 6-well plate. Cells grew for 5-7 days until they formed large colonies. PBS-EDTA solution was prepared as described above.

Generation of Adipocytes From bESCs

Culture of bESCs according to Method 1 and 2 for more than 5 days leads to ESC spontaneous formation of spheres which initially attached to the plate, but eventually became disconnected from matrix and freely float in the medium. For adipocyte differentiation, spheres were collected from medium by soft centrifugation at 50 g for 3 min. Pellets of spheres were resuspended and treated with enzyme free solution LeReSR (Stemcell, Cat #07174) at 37° C. for 3.5 min and diluted with cold mTeSR™plus medium. Dissociated cells are plated at a density of 5-7×10³/cm² onto collagen-precoated 6 wells plate in serum-free Gibco StemPro™ MSC SFM. Following several passages cells acquired typical adipocyte morphology and began expressing CD73. In Method 3, dissociated bESCs were plated at density 5-7×10³/cm² onto collagen-precoated 6 wells plate in serum-free Gibco StemPro™ MSC SFM. Following one passage cells, acquired typical adipocyte morphology and accumulated lipids.

### Evaluation of Expression of Pluripotency Markers Oct4 and Sox2

For immunohistochemistry, cells growing in 12 well plate were fixed in 2% formaldehyde in PBS (pH 7) for 10 min at room temperature (RT), washed with 3 times in MACS buffer, and incubated 50-60 min at RT with Permeabilization Buffer from Invitrogen (Ref #00-8333-56) diluted 1:10 with distilled water and supplemented with 5% Fetal Bovine Serum and 5% Donkey Serum, and then washed 3 times in MACS buffer. Primary Oct4 antibodies (OriGene Cat #AP23147—rabbit polyclonal antibody) was diluted 1:100 in MACS buffer and 150 mL was added to each well of 12 wells plate. After 1 hr incubation at RT, wells with cells washed out 3 times in MACS buffer and incubated for 50-60 min at RT with 150 mL/well FITC-conjugated anti-rabbit IgG antibody (OriGene, Cat #TA130021) diluted 1:50 in the MACS buffer with DAPI 2 mg/mL. Sox2 was detected using a similar procedure and a goat polyclonal SOX2 antibody (MyBioSource, Cat #MBS423840) and IgG (H+L) Cross-Adsorbed Rabbit anti-Goat, Alexa Fluor™ 488 (Invitrogen, Cat #A11078).

### Alkaline Phosphatase (AP) Detection

For detection of AP, bESCs were fixed in 2% of formaldehyde in PBS (pH 7) for 30-60 seconds at RT and washed 3 times in MACS buffer. Detection of AP was performed with kit from Vector Lab (Cat #SK-5300) subsequently mixed in 0.1 M TRIS buffer (pH 8).

### Adipocytes Staining

Fat accumulation in adipocytes was detected using Oil Red O dye. Oil Red O dye was dissolved in 10 mL of isopropanol and used as a stock solution. A working solution of Oil Red O was prepared by mixing 3 parts of stock of Oil Red O and 2 parts of deionized water and filtered. After mixing the working solution is stable for 2 hrs. Cells were fixed with 4% formaldehyde in PBS for 10 min, washed twice with the MACS buffer and incubated with 1.5-2 mL 60% isopropanol for 3-5 min. After aspiration of isopropanol, the working solution of Oil Red O was added to cells. Nuclei were stained with DAPI (2 mg/mL) and observed under microscope with DAPI and TRITC filters.

### CD73 Detection

Marker CD73 is one of the classic markers that specifically defines the mesenchymal stem/stromal cells (MSC) populations and is located on cell membrane. For CD73 detection, cells were fixed for 5 min in 2% formaldehyde in PBS and washed 3 times with the MACS buffer and incubated for 1 hr with 10% FBS in PBS to block nonspecific binding. After washing, cells were incubated for 40-50 min with an anti-CD73 rabbit polyclonal antibody (Bios, Ref #bs-4834r; www.biossusa.com/products/bs-4834r) diluted 1:150 and washed 3 times with the MACS buffer. 150 mL/well of Alexa 594 conjugated secondary antibody (Invitrogen Cat #A32740 Goat anti-Rabbit IgG (H+L) Highly Cross-Adsorbed Secondary Antibody) diluted to 1:100 in MACS buffer with 2 mg/mL DAPI was added to each well. Cells were incubated for 50-60 min at RT and washed 2 times in MACS buffer and observed under microscope with DAPI and TRITC filters.

### ADIPOQ and Leptin Detection

ADIPOQ (adiponectin) and Leptin are adipocyte markers. For detection of adiponectin and leptin, cells were fixed in 4% formaldehyde, washed 3 times by MACS buffer and permeabilized 30 min at RT using permeabilization buffer (Invitrogen, Ref #00-8333-56) and stained with rabbit polyclonal anti-LEP (MBS2028770, MyBiosource) or anti-ADIPOQ (adiponectin) (MBS8241984, MyBiosource) diluted 1:75 and washed 3 times with MACS buffer. Goat anti-rabbit polyclonal secondary antibody labeled with Alexa Fluor™ Plus 594 (Cat #A32740, Invitrogen) was applied after dilution 1:150 with MACS buffer for 1 hr at RT. After labeling nuclei with Hoechst 33542, cells were observed under fluorescent microscope.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the scope of the present invention, as defined in the following claims.

What is claimed is:

1. A method of culturing bovine embryonic stem cells (bESCs), the method comprising:
culturing bESCs in a serum-free culture medium comprising an effective amount of a γ-secretase inhibitor on a matrix-coated culture vessel in feeder-free conditions; and
obtaining an expanded population of bESCs expressing Oct4, Sox2, and alkaline phosphatase.

2. The method of claim 1, wherein the γ-secretase inhibitor comprises (2S)-N-[(3,5-Difluorophenyl)acetyl]-L-alanyl-2-phenyl]glycine 1,1-dimethylethyl ester (DAPT).

3. The method of claim 1, wherein the matrix comprises vitronectin or collagen.

4. A method of producing adipocytes from bovine embryonic stem cells (bESCs), the method comprising:
(a) culturing bESCs in a serum-free culture medium on a matrix-coated cell culture vessel in feeder-free conditions to obtain bESCs expressing Oct4, Sox2, and alkaline phosphatase, wherein the cell culture medium comprises an effective amount of γ-secretase inhibitor; and
(b) dissociating the bESCs and culturing the dissociated cells on a matrix-coated cell culture vessel under serum-free medium to obtain adipocytes.

5. The method of claim 4, wherein the γ-secretase inhibitor comprises DAPT.

6. The method of claim 4, wherein the serum-free culture medium in step (a) is mTeSR medium.

7. The method of claim 4, wherein the matrix comprises vitronectin or collagen.

8. The method of claim 4, wherein the dissociated cells are cultured in serum-free medium for 3-5 passages.

9. The method of claim 4, wherein the serum-free medium in step (b) is Gibco StemPro™ MSC SFM.

10. The method of claim 4, wherein the adipocytes exhibit adipocyte morphology and accumulate lipids.

11. The method of claim 10, wherein the adipocytes express adiponectin and leptin.

12. A method of producing adipocytes from bovine embryonic stem cells (bESCs), the method comprising:
(a) culturing blastocysts on mouse embryonic fibroblasts (MEFs) in a serum-free culture medium comprising an effective amount of one or more inhibitors of the WNT pathway to obtain bESCs expressing Oct4, Sox2, and alkaline phosphatase;
(b) culturing the bESCs from step (a) on a matrix-coated cell culture vessel in feeder-free conditions to form bESC spheres; and
(c) dissociating cells from the bESC spheres and culturing the dissociated cells on a matrix-coated cell culture vessel under serum-free medium to obtain adipocytes.

13. The method of claim 12, wherein the blastocysts are cultured on MEFs for 12-14 days.

14. The method of claim 12, wherein the one or more inhibitors of the WNT pathway comprises IWR-1, CCT251545, and/or iCRT3.

15. The method of claim 12, wherein the culture medium further comprises an Lck/Src inhibitor, Activin A, and LIF.

16. The method of claim 15, wherein the Lck/Src inhibitor comprises WH-4-023.

17. The method of claim 12, wherein the matrix comprises vitronectin or collagen.

18. The method of claim 12, wherein the bESCs in step (b) are cultured for about 5 days.

19. The method of claim 12, wherein the dissociated cells are cultured in serum-free medium for 3-5 passages.

20. The method of claim 12, wherein the serum-free medium in step (c) is Gibco StemPro™ MSC SFM.

21. The method of claim 12, wherein the adipocytes exhibit adipocyte morphology and accumulate lipids.

22. The method of claim 12, wherein the adipocytes express adiponectin and leptin.
